(19) United States
(12) Patent Application Publication
(10) Pub. No.: US 2003/0175898 A1
(43) Pub. Date: Sep. 18, 2003

(54) Biological material and uses thereof

(76) Inventor: Panagiotis Pantelidis, London (GB)

Correspondence Address: NEEDLE & ROSENBERG P C, 127 PEACHTREE STREET N E, ATLANTA, GA, 30303-1811, US

(21) Appl. No.: 10/204,653

(22) PCT Filed: Feb. 20, 2001
(86) PCT No.: PCT/GB01/00707

(51) Int. Cl. C12P 21/02; C07H 21/04; C12P 21/04; C12N 5/06; C07K 14/54

(52) U.S. Cl. 435/69.52; 435/70.21; 530/351; 536/23.5; 435/320.1; 435/325; 530/388.23

(57) ABSTRACT

The present invention provides an isolated nucleic acid molecule having a variation of the IL-13 encoding sequence shown in FIG. (1) wherein the variation is at least one of G to C at position +543nt and/or C to T at position +1922nt and/or G to A at position +2043nt and/or C to A at position +2579nt upstream of the initiation codon. The invention further provides an isolated amino acid sequence encoding a variant IL-13 containing glutamine at amino acid position 130, and the use of said amino acid sequence in a method of producing an antibody. Additionally, there is provided a method of detecting susceptibility or resistance to a disorder associated with an immune response comprising testing nucleic acid from an individual for the presence of a variation in the nucleotide sequence encoding IL 13.

This invention relates to variants of the nucleic acid sequence encoding Interleukin 13 (IL-13) and the use of such sequence variants in medicine, especially in the diagnosis of susceptibility or resistance to disorders associated with an immune response, particularly the inflammatory response associated with asthma, atopic allergy and latex sensitisation.

Numerous studies have demonstrated that CD4⁺T lymphocytes, via the release of specific cytokines, regulate the inflammatory response observed in asthma (for example, see Robinson D, Hamid Q, Bentley A, Ying S, et al. (1993) Journal Of Allergy And Clinical Immunology 92: 313-24; Robinson D S, Ying S, Bentley A M, Meng Q, et al. (1993) Journal Of Allergy And Clinical Immunology 92: 397-403; Robinson D S, Hamid Q, is Ying S, Tsicopoulos A, et al. (1992) New England Journal of Medicine 326: 298-304; Ying S, Durham S R, Corrigan C J, Hamid Q, et al. (1995) American Journal Of Respiratory Cell And Molecular Biology 12: 477-87). The T helper cell type 2 (T_{H}2) cytokines, which include interleukin-4 (IL-4), IL-5 and IL-10 have been implicated in the development of allergic inflammation. High expression of these cytokines has been observed in the bronchoalveolar lavage (BAL) cells and bronchial biopsies of asthmatic patients (Robinson D, Hamid Q, Bentley A, Ying S, et al. (1993) Journal Of Allergy And Clinical Immunology 92: 313-24; Robinson D S, Ying S, Bentley A M, Meng Q, et al. (1993) Journal Of Allergy And Clinical Immunology 92: 397-403; Robinson D S, Hamid Q, Ying S, Tsicopoulos A, et al. (1992) New England Journal of Medicine 326: 298-304; Ying S, Durham S R, Corrigan C J, Hamid Q, et al. (1995) American Journal Of Respiratory Cell And Molecular Biology 12: 477-87). IL-13 is biologically closely related to IL4 and shares signal transduction elements as well as receptor components with IL4 (Punnonen J, Aversa G, Cocks B G, McKenzie A N J, et al. (1993) Proc Natl Acad Sci USA 90:3730-4; McKenzie A N J, Culpepper J A, Malefyt R D, Briere F, et al. (1993) Proc Natl Acad Sci USA 90: 3735-9; Sornasse T, Larenas P V, Davis K A, deVries J E, et al (1996) Journal of Experimental Medicine 184: 473-83; Lefort S, Vita N, Reeb R, Caput D, et al. (1995) FEBS Letters 366: 122-6). It is produced at high levels by CD4+T_{H}2 cells after activation but has also been found to be produced by other T cell subsets including T_{H}0 and CD8+T cells (De Waal Malefyt R D, Abrams J S, Zurawski S M, Lecron J C, et al. (1995) International Immunology 7: 1405-16). One of most important similarities with IL-4 is the ability to induce IgE production (Punnonen J, Aversa G, Cocks B G, McKenzie A N J, et al. (1993) Proc Natl Acad Sci USA 90:3730-4; McKenzie A N J, Culpepper J A, Malefyt R D, Briere F, et al. (1993) Proc Natl Acad Sci USA 90: 3735-9; Emson C L, Bell S E, Jones A, Wisden W, et al. (1998) Journal of Experimental Medicine 188:399404; Dolecek C, Steinberger P, Susani M, Kraft D, et al. (1995) Clinical And Experimental Allergy 25:879-89; Punnonen J, Yssel H, deVries J E (1997) Journal Of Allergy And Clinical Immunology 100:792-801). However, unlike IL-4, IL-13 is ineffective in directing T_{H}2-cell differentiation (Sornasse T, Larenas P V, Davis K A, deVries J E, et al. (1996) Journal of Experimental Medicine 184: 473-83).

Evidence suggesting a critical role for IL-13 in asthma comes from a well-characterised experimental murine model of allergic asthma (WillsKarp M, Luyimbazi J, Xu X Y, Schofield B, et al. (1998) Science 282:2258-61; Grunig G, Warnock M, Wakil A E, Venkayya R, et al. (1998) Science 282:2261-3). Sensitization and subsequent challenge of mice with allergen results in airway hyperresponsiveness, eosinophil recruitment, increase in specific IgE, and mucus overproduction. Selective neutralization of IL-13 in these models ameliorates the asthma phenotype through a reduction in airway hyperresponsiveness, mucus secretion and BAL eosinophilia. Daily administration of IL-13 to the airways of naïve mice was shown to be sufficient to induce airway hyperresponsiveness, BAL eosinophilia, increased total serum IgE, and goblet cell metaplasia with mucus overproduction (WillsKarp M, Luyimbazi J, Xu X Y, Schofield B, et al. (1998) Science 282:2258-61; Grunig G, Warnock M, Wakil A E, Venkayya R, et al. (1998) Science 282:2261-3). Similarly, the selective expression of IL-13 in the lung of transgenic mice has been shown to cause a mononuclear and eosinophilic inflammatory response, mucus hypersecretion, subepithelial fibrosis, non-specific airway hyperresponsiveness, and increased production of the eosinophil chemoattractant eotaxin (Zhu Z, Homer R J, Wang Z, Chen Q, et al. (1999) J Clin Invest 103:779-88). In humans, increased expression of IL-13 has been observed in bronchial biopsies from atopic asthmatics (Naseer T, Minshall E M, Martin R J, Laberge S, et al. (1997) American Journal Of Respiratory And Critical Care Medicine 155:845-51) and peripheral blood mononuclear cells from atopic patients (Esnault S, Benbernou N, Lavaud F, Shin H C, et al. (1996) Clinical And Experimental Immunology 103:111-8).

The human IL-13 gene is located on chromosome 5q31, approximately 12 kb upstream from the IL-4 gene. Large-scale familial linkage studies have linked this region of chromosome 5 to allergy and asthma susceptibility (Palmer L J, Daniels S E, Rye P J, Gibson N A, et al. (1998) American Journal Of Respiratory And Critical Care Medicine 158:1825-30; Rosenwasser L J (1998) Allergy 53:8-11; Noguchi E, Shibasaki M, Arinamni T, Takeda K, et al. (1997) American Journal Of Respiratory And Critical Care Medicine 156:1390-3; Bleecker E R, Postma D S, Meyers D A (1997) CIBA Foundation Symposia 206:90-105). Recently, Anderson et al reported that using single stranded conformational polymorphism analysis (SSCP-PCR), no polymorphisms in the promoter region spanning from nucleotide −1039 (−1039nt) to +80nt were found (Anderson K L, Mathieson P W, Gillespie K M (1999) Science 284: 1431a). The absence of polymorphisms in the promoter region of IL-13 was also confirmed by the reply to that correspondence by M. Wills-Karp and L. J. Rosenwasser which also examined the IL-13 putative promoter region for the presence of polymorphisms.

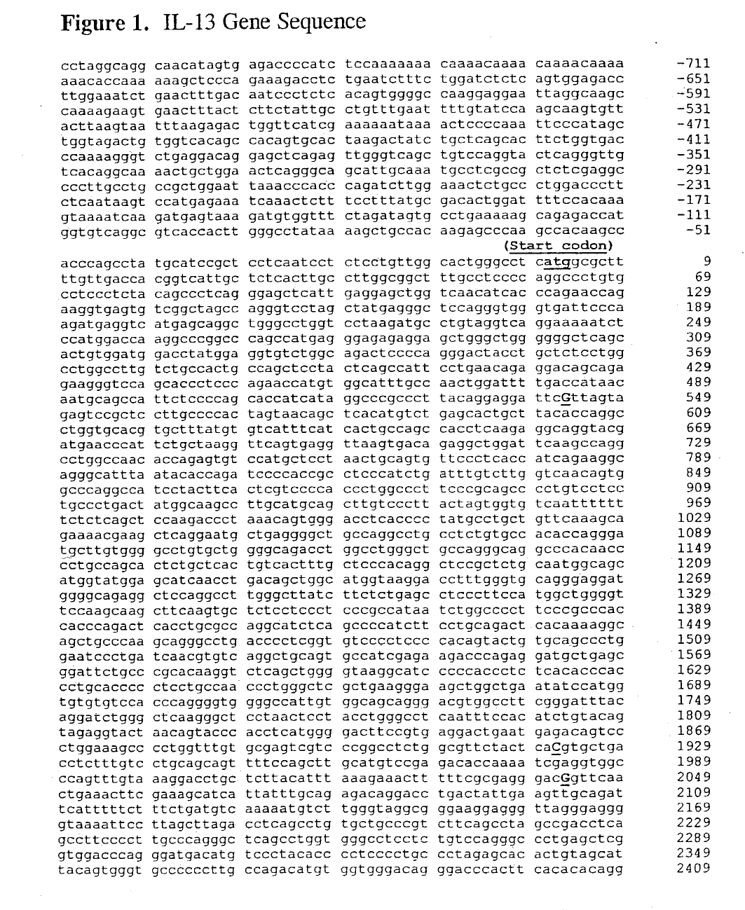

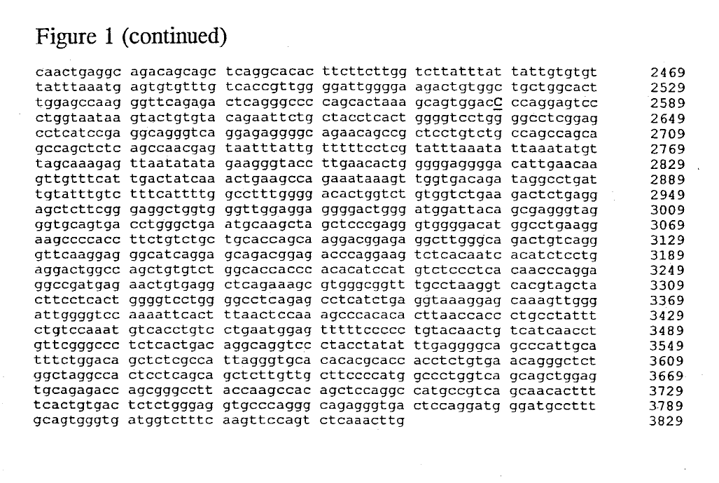

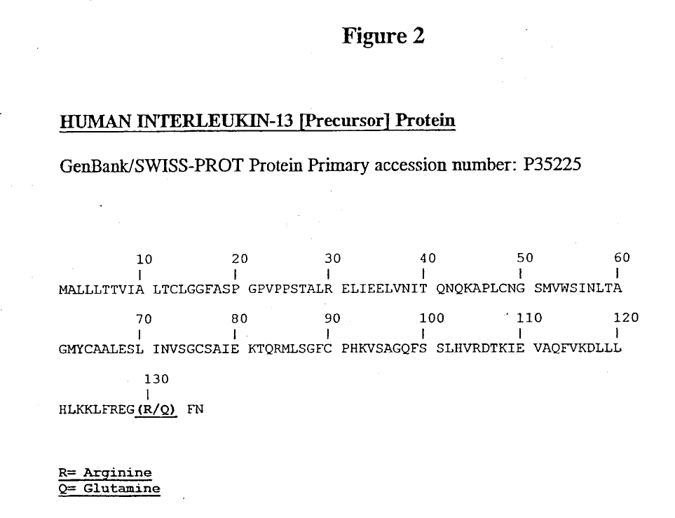

Unexpectedly, by comparing the IL-13 gene sequences deposited in the GenBank™ database, upstream of nucleotide +80, we identified four single nucleotide variations in four of the deposited sequences of the IL-13 gene. The four potential single nucleotide polymorphisms (SNP's) were: a G/C at +543nt, a C/T at +1922nt, a G/A at +2043nt and a C/A at +2579nt upstream of the first nucleotide of the start codon (FIG. 1; [SEQ ID No 1]), which represent nucleotide positions 1314, 2693, 2814 and 3350 respectively in GenBank™ deposited sequence L13029. The variations at positions +543nt and +1922nt were located in introns 1 and 3, respectively, whereas the variations at positions +2043nt and +2579nt were located in the translated and 3′-untranslated regions of exon 4, respectively. Moreover, the G to A substitution at position +2043nt was found to change the codon sequence CGC that codes for the basic amino acid arginine (Arg) at amino acid position 130 of the unprocessed precursor (see GenBank™ deposited sequence P35225), to CAG that codes for the hydrophilic amino acid glutamine (Gln) (see FIG. 2; [SEQ ID No 2]).

In a first aspect the invention provides an isolated nucleic acid molecule having a variation of the IL-13 encoding sequence shown in FIG. 1 [SEQ ID No 1]; wherein the variation is at least one of G to C at position +543nt and/or C to T at position +1922nt and/or G to A at position +2043nt and/or C to A at position +2579nt upstream of the initiation codon.

Preferably, the variation is G to A at position +2043nt. More preferably, the variation is C to T at position +1922, G to A at position +2043 and C to A at position +2579.

The invention also provides a nucleic acid molecule according to this aspect of the invention for use in medicine.

A second aspect the invention provides an isolated amino acid sequence encoded by a nucleic acid molecule according to this aspect of the invention and comprising glutamine at an amino acid position corresponding to position 130 of the unprocessed precursor (see FIG. 2; SEQ ID No 2).

Preferably, said amino acid sequence encoded by a nucleic acid molecule according to this aspect of the invention has IL-13 activity.

By “isolated” as used in relation to the first and second aspects of the invention we include the meaning that the material is free of at least some of the biological substances with which it exists in nature. However, the material of the invention may of course be provided as a composition containing other materials with which it does not exist in nature, and such compositions are intended to fall within the scope of the invention.

By “IL-13 activity” we include the meaning that the amino acid sequence has at least one of the functional properties attributed to naturally-occurring (i.e. wildtype) IL-13. Preferably, the amino acid sequence with IL-13 activity is capable of one or more of the following:

(i) Induction of IgE synthesis by unfractionated peripheral blood mononuclear cells (PBMNC) and anti-CD-40 stimulated B-cells, as measured by ELISA (see Dolecek et al., 1995, Clin. Exp. Allergy 25:879-89; Levy et al., 1997, Int. Arch. Allergy Immunol. 112:49-58);

(ii) Inhibition of LPS-stimulated production of nitric oxide (NO) by macrophages (see Bogdan et al., 1997, J. Immunol. 159:4506-13; Doherty et al., 1993, J. Immunol. 151:7151-60);

(iii) Modification of cell surface markers on adherent cells (e.g. monocytes) from peripheral blood (Morse et al., 1999, J. Immunother. 22:506-13); and

(iv) Proliferation of B-cells (McKenzie et al., 1993, Proc. Natl. Acad. Sci. USA 90:3735-3739)

Additionally, IL-13 activity may be assessed using either in vitro or in vivo systems by measuring the ability of the amino acid sequence to bind to naturally occurring IL-13 receptors and/or to modulate cellular events associated with binding of IL-13 to said IL-13 receptors (for example, see Debinski et al., 1998, Int. J. Cancer 76:547-51; Debinski et al., 1996, J. Biol. Chem. 271:22428-33; Obiri et al., 1996, Clin. Cancer Res. 2:1743-9; Debinski et al., 1995, Clin. Cancer Res. 1:1253-8).

A third aspect of the invention provides a transgenic, non-human mammalian animal whose germ cells and somatic cells contain a nucleic acid molecule according to the first aspect of the invention. Preferably, the transgenic animal is capable of expressing an amino acid sequence having IL-13 activity and containing glutamine at amino acid position 130.

By “transgenic” we mean the animal has a foreign nucleic acid construct inserted into its genome. It will be appreciated that, in principle, the transgenic animal may be from any species of non-human mammalian animal, such as rats, mice, rabbits, cattle, sheep, and pigs.

A further aspect of the invention provides a method of producing a transgenic non-human mammalian animal according to the third aspect of the invention, said method comprising introducing a nucleic acid molecule according to the first aspect of the invention into a non-human mammalian animal, preferably at a stage no later than the 8-cell stage.

Various methods for creating transgenic animals are known in the art. The principal means by which transgenic animals are currently produced are: pronuclear DNA microinjection; blastocyst microinjection of embryonic stem (ES) cells; and replication-defective viral vector transduction (Jaenisch, R., 1988, Science 240, 1468-1474).

Human embryonic stem (ES) cells may be used to produce a transgenic animal containing coamplified copies of the gene of interest by established procedures (Robertson, E. J, 1987, Teratomas and embryonic stem cells: a practical approach, IRL Press, Oxford, U.K.). The ES system has been developed in the mouse, but is directly applicable to other animal species where ES cells can be isolated. Briefly, chimaeric animals are produced, either by injecting ES cells into host blastocysts, or by aggregating ES cells with host morulae. In each case, the chimaeric embryos are reimplanted into foster mothers and allowed to develop into chimaeric animals. If the ES cells have contributed to the germ line of the chimaera, then some gametes from the chimaera will be ES cel-derived. By crossing a chimaera with another animal, progeny with ES cell-derived genetic material can be obtained. If the ES cells used contain co-amplified copies of the gene of interest, some of the progeny will contain the co-amplified gene in every cell of their bodies. In this way transgenic strains containing the co-amplified gene can be established.

A second method of producing transgenic animals, which is likely to be particularly valuable in larger mammalian species, such as sheep and cattle may also be used to generate a transgenic animal of the present invention. The basic procedure has been described for the cloning of sheep (Campbell, K. H. S., McWhir, J., Ritchie, W. A. and Wilmut, 1996, Nature 380:64-66; Wilmut, I., Schnieke, A. E., McWhir, J., Kind, A. J. and Campbell, K. H. S., 1997, Nature 385: 810-813).

Briefly, a cell line was established from a day 9 sheep embryonic disc. Nuclear transfer from these cells into enucleated oocytes resulted in the production of viable lambs. The procedure was subsequently repeated using nuclei from foetal fibroblasts and, in one case, from adult mammary epithelial cell cultures. Isolation of cells with little or no expression of a given selectable protein from derivatives of such cell lines, derived from early animal embryos, foetuses, or adult tissues and which retain totipotency for nuclear transfer, will permit the production, by nuclear transfer into enucleated oocytes, of transgenic animals containing co-amplified copies of a gene of interest.

See also, WO 97/07669, WO 98/30683 and Sims et al. (1993), Proc. Natl. Acad. Sci. USA 90:6143-6147 for further information on the production of transgenic animals using nuclear transfer protocols.

A transgenic animal of the present invention may be a chimaera or it may express multiple copies of a gene of interest in all its somatic cells. Also, a transgenic animal of the present invention may be a first generation transgenic animal or any of its progeny which comprise multiple copies of the gene of interest.

Preferably, a transgenic animal of the present invention expresses substantial amounts of the gene product of interest (i.e. a variant amino acid sequence with IL-13 activity), either constitutively or in a regulated manner, throughout the entire body or restricted to a particular tissue or body fluid.

Methods for achieving the tissue-specific expression of a transgene are amply described in the art. For example, the metallothionein promoter has been used to direct the expression of the rat growth hormone in the liver tissue of transgenic mice (Palmiter et al (1982), Nature 300:611). Another example is the elastase promoter, which has been shown to direct the expression of foreign genes in the pancreas (Ornitz et al (1985), Nature 313:600). See also EP 279 582, which describes methods for the targeting of proteins to the mammary gland and the subsequent secretion of biologically important molecules in the milk.

Developmental control of gene expression has also been achieved in transgenic animals, i.e. the foreign gene is transcribed only during a certain time period, and only in a certain tissue. For example, Magram et al (1985 Nature 315:338) demonstrate the developmental control of genes under the direction of a globin promoter.

Proteins produced by a transgenic animal of the present invention may then be harvested e.g. from its serum, milk or ascites fluid. The desired protein may then purified from other host proteins by methods well known in the art to obtain preparations of the desired protein that are substantially homogeneous.

It will be understood by those skilled in the art that transgenic animals according to the third aspect of the invention may have utility in screening assays for identifying candidate compounds with efficacy in the treatment of immune disorders, such as asthma, atopic allergies and latex sensitisation. Thus, the present invention provides a method of screening for candidate compounds with efficacy in the treatment of immune disorders comprising:

(i) administering a compound to be tested to a transgenic animal according to the third aspect of the invention; and

(ii) measuring a biological marker of immune system function or dysfunction in said animal.

Preferably, candidate compounds will be selected which increase markers associated with immune system function and/or decrease markers associated with immune system dysfunction.

Suitable biological markers include phenotypic markers of immune system disease states. For example, Symula et al. (1999) Nature Genetics 23:241-244 discloses the measurement of asthma phenotype markers (specifically serum IgE, maximum bronchoconstrictor response and bronchoalveolar lavage eosinophilia) in transgenic mice containing a 1 Mb sequence from chromosome 5q31.

In a fourth aspect, the invention provides the use of an amino acid sequence according to the second aspect of the invention in a method of producing an antibody.

The antibody may be a polyclonal antibody, but is preferably a monoclonal antibody.

A fifth aspect of the invention also provides an antibody obtainable by a use of the above method, wherein the antibody specifically binds the amino acid sequence according to the second aspect of the invention and does not exhibit significant cross-reactivity with a different IL-13 encoding amino acid sequence.

The invention also provides the amino acid sequence of the second aspect of the invention for use in medicine.

The invention further provides a method of detecting susceptibility or resistance to a disorder associated with expression of IL-13 comprising testing nucleic acid from an individual for the presence or absence of a variation in the nucleotide sequence encoding IL-13 as defined in accordance with the first aspect of the invention.

The invention further provides a method of detecting susceptibility or resistance to a disorder associated with expression of IL-13 comprising testing a biological sample from an individual for the presence or absence of an amino acid sequence as defined in accordance with the second aspect of the invention.

Preferably, the amino acid sequence is detected using an antibody.

Preferably, the disorder is associated with an immune response and is preferably asthma and/or latex sensitisation.

The invention also provides an antibody obtainable by use or method as defined previously for use in medicine.

The invention still further provides a method of detecting susceptibility or resistance to a disorder associated with an immune response comprising testing nucleic acid from an individual for the presence of a variation in the nucleotide sequence encoding IL-13 as defined in accordance with the first aspect of the invention.

Preferably, the invention provides a method of detecting susceptibility or resistance to latex sensitisation of an individual comprising testing nucleic acid from the individual for the presence or absence of a variation in the nucleotide sequence encoding IL-13 as defined in accordance with the first aspect of the invention, the presence of such a variation being indicative of latex sensitivity.

A further aspect of the invention provides a method of treatment of a patient with an immune response disorder comprising administering to said patient a blocking agent which binds to a nucleic acid molecule according to the first aspect of the invention and/or to an amino acid sequence according to the second aspect of the invention, thereby preventing or reducing the expression of said nucleic acid molecule and/or preventing or reducing the function of said amino acid sequence.

Preferably, the patient with an immune response disorder is suffering from asthma or latex sensitisation.

Suitable blocking agents include antisense oligonucleotides and antibodies.

Antisense oligonucleotides are single-stranded nucleic acids, which can specifically bind to a complementary nucleic acid sequence. By binding to the appropriate target sequence, an RNA-RNA, a DNA-DNA, or RNA-DNA duplex is formed. These nucleic acids are often termed “antisense” because they are complementary to the sense or coding strand of the gene Recently, formation of a triple helix has proven possible where the oligonucleotide is bound to a DNA duplex. It was found that oligonucleotides could recognise sequences in the major groove of the DNA double helix. A triple helix was formed thereby. This suggests that it is possible to synthesise a sequence-specific molecules which specifically bind double-stranded DNA via recognition of major groove hydrogen binding sites.

By binding to the target nucleic acid, the above oligonucleotides can inhibit the function of the target nucleic acid. This could, for example, be a result of blocking the transcription, processing, poly(A)addition, replication, translation, or of promoting inhibitory mechanisms of the cells such as RNA degradation (for example, see Goodchild, 1989, In: Oligonucleotide antisense inhibitors of gene expression, Cohen J S (Ed.), Macmillan Press, pp 53-77; Milligan J F et al., 1993, J. Med. Chem. 36:1923-1937; Ross J, 1988, Mol. Biol. Med. 5:1-14; Stein C A et al., 1988, Nucleic Acids Res. 16:3209-3221; Uhlman E & Peyman A, 1990, Chemical Rev. 90:543-584; Walder R Y & Walder J A, 1988, Proc. Natl. Acad. Sci. USA 85:5011-5015).

Typically, antisense oligonucleotides are 15 to 35 bases in length. For example, 20-mer oligonucleotides have been shown to inhibit the expression of the epidermal growth factor receptor mRNA (Witters et al, Breast Cancer Res Treat 53:41-50 (1999)) and 25-mer oligonucleotides have been shown to decrease the expression of adrenocorticotropic hormone by greater than 90% (Frankel et al, J Neurosurg 91:261-7 (1999)). However, it is appreciated that it may be desirable to use oligonucleotides with lengths outside this range, for example 10, 11, 12, 13, or 14 bases, or 36, 37, 38, 39, 40 or more bases.

Preferably the blocking agent is an antisense oligonucleotide complementary in sequence to a nucleic acid molecule according to the first aspect of the invention.

The antisense oligonucleotides may be administered systemically. Alternatively, the oligonucleotides can be delivered to a specific locus by any means appropriate for localised administration of a drug. For example, a solution of the oligonucleotides can be injected directly to the site or can be delivered by infusion using an infusion pump. The oligonucleotides can also be incorporated into an implantable device which when placed at the desired site, permits the oligonucleotides to be released into the surrounding locus.

The dose of oligonucleotide and the administration protocol used to deliver it will be optimised so as to maximise the therapeutic effect (e.g. the positive effect on immune system function and/or the negative effect on immune system dysfunction) and minimise the unwanted side-effects. Optimsation of antisense therapies is discussed in Kairemo K J et al. (2000) Methods Enymol. 314:506-524.

Preferably, the antisense oligonucleotides are targeted to T_{H}2 cells.

The oligonucleotides may be administered to the patient systemically for both therapeutic and prophylactic purposes. The oligonucleotides may be administered by any effective method, for example, parenterally (e.g. intravenously, subcutaneously, intramuscularly) or by oral, nasal or other means which permit the oligonucleotides to access and circulate in the patient's bloodstream. Oligonucleotides administered systemically may be given in addition to locally administered oligonucleotides, but also have utility in the absence of such local administration.

Advantageously, the blocking agent is an antibody according to the fifth aspect of the invention.

The various materials and methods of the invention are suitable for use in medicine, preferably in the prevention, treatment and/or diagnosis of a disorder associated with expression of IL-13 such as latex sensitisation and/or asthma. It will be appreciated that other such disorders are intended to fall within the scope of this invention.

Preferred non-limiting examples embodying certain aspects of the invention will now be described:

FIG. 1 shows the nucleotide sequence of the IL-13 gene, as specified in GenBank sequence accession number L13029 (the numbering of the nucleotides is altered, however). The nucleotides are numbered from the first nucleotide of the start codon, which is designated nucleotide 1 (this nucleotide corresponds to nucleotide 771 in the L13029 sequence [SEQ ID No 1]).

FIG. 2 shows the amino acid sequence of the IL-13 precursor [SEQ ID No 2]. The signal sequence comprises residues 1 to 20, and the mature peptide comprises residues 21 to 132.

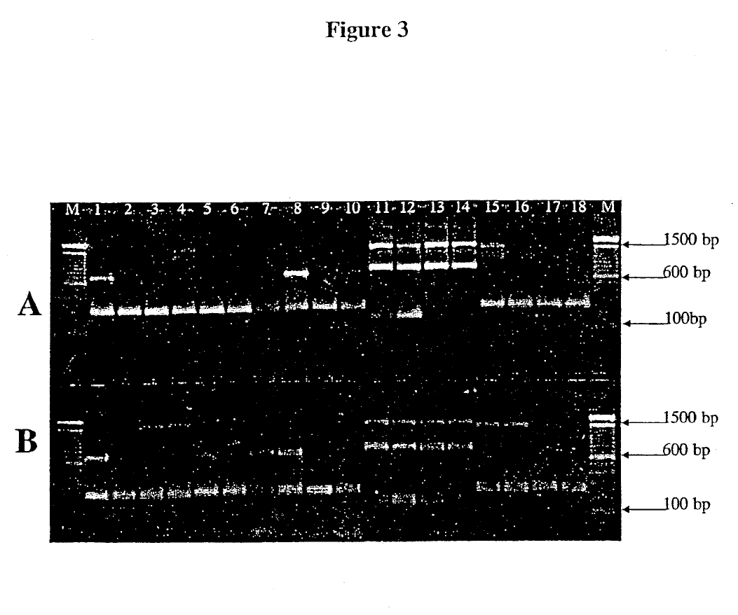

FIG. 3 shows exemplary data using two DNA samples that underwent PCR amplification using the allele specific primer mixtures 1 to 18, as described in Table 2. The PCR products were separated in a 2% agarose gel; M=100 bp DNA ladder (Life Technologies Ltd, Paisley, UK). DNA sample A produced positive reactions with primer mixtures 1, 4, 8, 12 and 15, indicating that this sample is from an individual who is homozygous for alleles G, C, G and C at positions +543nt, +1922nt, +2043nt and +2579nt, respectively. DNA sample B produced positive reactions with primer mixtures 1, 3, 4, 7, 8, 12, 13, 15 and 16, indicating that this sample is from an individual who is heterozygous for alleles (C/T), (G/A) and (C/A) at positions +1922nt, +2043nt and +2579nt, respectively. From the primer combinations, together with the haplotype nomenclature of Table 4, it can be deduced that DNA sample A is from an individual with the AA genotype whereas that DNA sample B is from an individual with the AB genotype.

### METHODS AND EXAMPLES

To confirm the existence of these potential polymorphisms we used Sequence Specific Primer-PCR (SSP-PCR) methodology, which has been used previously to characterize SNP's in the tumour necrosis factor-α and lymphotoxin-α genes (Fanning G C, Bunce M, Black C M, Welsh K I (1997) Tissue Antigens 50: 23-31). We designed sequence specific primers with 3′-end mismatches identifying each of the variants at the four polymorphic sites (table 1) and we used the specific primers to identify the individual variants by PCR amplification (table 2), as previously described (Bunce M, O'Neill C M, Barnardo C N M, Krausa P, et al. (1995) Tissue Antigens 46: 355-367). An appropriate set of control primers added to all reactions (tables 1 and 2) confirmed PCR amplification where the variant was absent. Using the DNA from an initial population of 50 UK controls, we were able to confirm that the single nucleotide variations at the four sites identified by sequence comparisons were genuine and not sequencing errors (FIG. 3). Subsequently, we examined the frequency of all four SNP's in 196 UK Caucasoid controls in total. As experimental evidence suggests a critical role for IL-13 in allergy and asthma, we also examined the frequency of the four SNP's in a group of 26 subjects with well-characterised latex allergy (LTX).

1. All PCR reactions were carried out under identical conditions and as previously described for HLA phototyping in a final volume of 13 μl overlaid with 10 μl mineral oil (Bunce M, O'Neill C M, Barnardo C N M, Krausa P, et al. (1995) Tissue Antigens 46: 355-367). Each reaction consisted of 5 μl of the appropriate primer mix (Table 2) and 8 μl of PCR reaction mixture in 96-well plates (final concentrations of the constituents of the PCR reaction mixture were 1× PCR buffer (Bioline, London, UK), 160 μM of each dNTP (Bioline, London, UK), 2 mM MgCl₂, 0.3 U Taq polymerase (Bioline, London, UK) and 0.01-0.1 μg DNA). PCR amplifications were carried out in a MJ Research PTC-200 machine. The cycling parameters for 13 μl reactions were 96° C. for 1 min, followed by five cycles of 96° C. for 25 sec, 70° C. for 45 sec, 72° C. for 25 sec, then 21 cycles of 96° C. for 25 sec, 65° C. for 50 sec, 72° C. for 30 sec, followed by 4 cycles of 96° C. for 30 sec, 55° C. for 60 sec and 72° C. for 90 sec. To the completed PCR reaction, 10 μl of loading dye (Bunce M, O'Neill C M, Barnardo C N M, Krausa P, et al. (1995) Tissue Antigens 46: 355-367) were added and the entire product was loaded into a 2% agarose/10.5× TBE gel containing 0.5 μg/ml ethidium bromide. Electrophoresis was carried out for 20 min at 200 V/cm² and the gel was photographed under UV light (320 nm). The presence of an allele-specific band of the expected size in conjunction with a control band was considered to be positive evidence for each particular allele. The absence of an allele specific band and the presence of a control band were considered to be negative evidence for the presence of an allele.

2. DNA was extracted from peripheral blood collected in EDTA and was resuspended and stored in water. Unrelated UK control subjects were used in this study. All subjects were cadaveric renal allograft donors collected from around the UK by the Oxford Transplant Centre, Churchill Hospital, Oxford. The representative nature of this control population for UK Caucasians has previously been demonstrated in HLA genotyping studies (Bunce M, O'Neill C M, Barnardo C N M, Krausa P, et al. (1995) Tissue Antigens 46: 355-367).

3. The 26 individuals used in this study represented the total number of confirmed latex allergy Caucasoid individuals referred to two occupational allergy referral centres (Royal Brompton and Harefield NHS Trust and Birmingham Heartland Hospital) over the period of 1996 to 1998. Patients with latex allergy were UK Caucasoid, had specific IgE to latex and clinical symptoms ranging from upper respiratory, chest to urticaria.

4. SNP IL-13 allelic associations between different polymorphic sites were analysed using a test by the statistical analysis program KnowledgeSEEKER (Angoss Software, Guildford, UK). A pc value <0.05 corrected for multiple comparisons (according to the formula pc=1−(1−p)ⁿ, where pc is the corrected value, p the uncorrected value, and n the number of allelic comparisons) was considered significant.

5. The genotype, phenotype and gene pool frequencies of the haplotypes in the control and LTX populations were determined by direct counting.

6. Initially the relative distribution of genotypes in the control population and LTX group were compared and a p value was generated using a 2×13 contingency table and the Chi-square statistics. Following observation of significance, the individual genotypes and the haplotype frequencies in the population and gene pool were examined using a 2×2 contingency table and Woolf-Haldane analysis. Similarly, the control population and latex group frequencies of each allele in the each of the four polymorphic sites were compared using a 2×2 contingency table and Woolf-Haldane analysis. In all cases a p value greater than 0.05 was considered significant.

TABLE 1
Primer sequences used in this study to identify the specific
alleles and to amplify the ‘control DNA’ sections

| Primer | Identified | | |
|---|---|---|---|
| Number | Specific Allele | Sequence | |
| 001 | +543nt (G) | 5′-gCCCTTACAggAggATTCg | [SEQ ID No 3] |
| 002 | +543nT (C) | 5′-gCCCTTACAggAggATTCC | [SEQ ID No 4] |
| 003 | Consensus to +543nt | 5′-gCCATTgCAgAgCgg AgC | [SEQ ID No 5] |
| 004 | +1922nt (T) | 5′-gCCTCTggCgTTCTACTCAT | [SEQ ID No 6] |
| 005 | +1922nt (C) | 5′-CCTCTggCgTTCTACTCAC | [SEQ ID No 7] |
| 006 | +2043nt (A) | 5′-gCTTTCgAAgTTTCAgTTgAACT | [SEQ ID No 8] |
| 007 | +2043nt (G) | 5-′gCTTTCgAAgTTTCAgTTgAACC | [SEQ ID No 9] |
| 008 | +2579nt (A) | 5′-TTATTACCAgggACTCCTggT | [SEQ ID No 10] |
| 009 | +2579nt (C) | 5′-ATTACCAgggACTCCTggG | [SEQ ID No 11] |
| 010 | (Reverse) +1922nt (C) | 5′-Agg ACAAAgAggTCAgCA CG | [SEQ ID No 12] |
| 011 | (Reverse) +1922nt (T) | 5′-AggACAAAgAggTCAgCA CA | [SEQ ID No 13] |
| 063 | DRB exon 3 | 5′ TgCCAAgTggAgCACCCAA | [SEQ ID No 14] |
| 064 | DRB exon 4 | 5′ gCATCTTgCTCTgTgCAgAT | [SEQ ID No 15] |
| 210 | APC* | 5′ ATgATgTTgACCTTTCCAggg | [SEQ ID No 16] |
| 211 | APC* | 5′TTCTgTAACTTTTCATCAgTTgC | [SEQ ID No 17] |

*APC—human adenomatous polyposis coli

TABLE 2
Primer mix combinations used to identify the individual alleles
in each polymorphic site of the IL-13 gene and their cis/trans
chromosomal arrangement.

| | Primer No | Primer No | | | | | Allele | Control |
|---|---|---|---|---|---|---|---|---|
| | (A) | (B) | Identified Alleles at | | | | specific | PCR |
| Primer | [final conc | [final conc | polymorphic sites | | | | PCR product | product |
| mix | μM] | μM] | 543 | 1922 | +2043 | 2579 | (bp) | (bp) |
| 1 | 001 [0.66] | 003 [0.69] | G | | | | 682 | 256* |
| 2 | 002 [0.66] | 003 [0.69] | C | | | | 682 | 256* |
| 3 | 001 [0.79] | 006 [0.66] | G | | A | | 1541 | 256* |
| 4 | 001 [0.79] | 007 [0.66] | G | | G | | 1541 | 256* |
| 5 | 002 [0.79] | 006 [0.66] | C | | A | | 1541 | 256* |
| 6 | 002 [0.79] | 007 [0.66] | C | | G | | 1541 | 256* |
| 7 | 004 [0.76] | 008 [0.78] | | T | | A | 697 | 256* |
| 8 | 005 [0.81] | 009 [0.78] | | C | | C | 694 | 256* |
| 9 | 004 [0.58] | 009 [0.59] | | T | | C | 695 | 256* |
| 10 | 005 [0.61] | 008 [0.54] | | C | | A | 696 | 256* |
| 11 | 005 [0.54] | 006 [0.44] | | C | A | | 162 | 796** |
| 12 | 005 [0.54] | 007 [0.44] | | C | G | | 162 | 796** |
| 13 | 004 [0.51] | 006 [0.44] | | T | A | | 163 | 796** |
| 14 | 004 [0.51] | 007 [0.44] | | T | G | | 163 | 796** |
| 15 | 001 [0.66] | 010 [0.62] | G | C | | | 1417 | 256* |
| 16 | 001 [0.66] | 011 [0.62] | G | T | | | 1417 | 256* |
| 17 | 002 [0.66] | 010 [0.62] | C | C | | | 1417 | 256* |
| 18 | 002 [0.66] | 011 [0.62] | C | T | | | 1417 | 256* |

*Control PCR product using primer pair 210-211 at a final concentration of 1 μM.
**Control PCR product using primer pair 63/64 at a final concentration of 0.2 μM

Final concentration for each primer refers to the concentration in the 13 μl reaction volume.

TABLE 3
Nomenclature of the detected haplotypes

Allele in each polymorphic position

| Haplotype | +543 | +1922 | +2043 | +2579 |
|---|---|---|---|---|
| A | G | C | G | C |
| B | G | T | A | A |
| C | C | C | G | C |
| D | C | T | A | A |
| E | G | C | G | A |
| F | G | T | G | C |
| G | G | T | G | A |
| H | C | T | G | A |

TABLE 4
Frequencies of the Haplotype in the control and latex group

| | |
|---|---|
| Control population | LATEX allergy |

| GENOTYPE. | Count | Frequency | Count | Frequency |
|---|---|---|---|---|
| AA | 130 | 0.66 | 11 | 0.42* |
| AB | 41 | 0.21 | 8 | 0.31 |
| BB | 3 | 0.02 | 1 | 0.04 |
| AC | 2 | 0.01 | 0 | 0.00 |
| AD | 10 | 0.05 | 2 | 0.08 |
| BD | 3 | 0.02 | 0 | 0.00 |
| AE | 1 | 0.01 | 0 | 0.00 |
| AF | 2 | 0.01 | 0 | 0.00 |
| AG | 2 | 0.01 | 1 | 0.04 |
| AH | 2 | 0.01 | 0 | 0.00 |
| FG | 0 | 0.00 | 1 | 0.04 |
| FH | 0 | 0.00 | 1 | 0.04 |
| DD | 0 | 0.00 | 1 | 0.04 |
| Total | 196 | | 26 | |

Phenotype frequencies

| | | | | |
|---|---|---|---|---|
| A | 190 | 0.97 | 22 | 0.85* |
| B | 47 | 0.24 | 9 | 0.35 |
| C | 2 | 0.01 | 0 | 0.00 |
| D | 13 | 0.07 | 3 | 0.12 |
| E | 1 | 0.01 | 0 | 0.00 |
| F | 2 | 0.01 | 2 | 0.08* |
| G | 2 | 0.01 | 2 | 0.08* |
| H | 2 | 0.01 | 1 | 0.04 |

Allele frequencies

| | | | | |
|---|---|---|---|---|
| A | 320 | 0.82 | 33 | 0.63* |
| B | 50 | 0.13 | 10 | 0.19 |
| C | 2 | 0.01 | 0 | 0.00 |
| D | 13 | 0.03 | 4 | 0.08 |
| E | 1 | 0.00 | 0 | 0.00 |
| F | 2 | 0.01 | 2 | 0.04* |
| G | 2 | 0.01 | 2 | 0.04* |
| H | 2 | 0.01 | 1 | 0.02 |

The genotype phenotype and allele frequencies were determined by direct counting. No significant deviation from Hardy Weinberg frequencies were observed (p>0.05).
*Indicates significant difference from the control population.

TABLE 5
Frequency of the individual alleles in the four
polymorphic sites of IL-13 gene.

| | | Control | Latex allergy |
|---|---|---|---|
| polymorphic | | n = 196 | n = 26 |
| position | Allele | Allele count (%) | Allele count (%) |
| +543 | G | 375 (95.6) | 47 (90.4) |
| | C | 17 (4.3) | 5 (9.6) |
| +1922 | C | 323 (82.4) | 33 (63.5)* |
| | T | 69 (17.6) | 19 (36.5)* |
| +2043 | G | 329 (83.9) | 38 (73.1)* |
| | A | 63 (16.1) | 14 (26.9)* |
| +2579 | C | 324 (82.7) | 35 (67.3)* |
| | A | 68 (17.3) | 17 (32.7)* |

*Indicates significant difference from the control population

We observed a strong linkage (M. C. Peitsch (1996) Biochem Soc Trans 24, 274.) between the presence of G allele at position +543, the presence of a C allele at position +1922, the presence of a G allele at position +2043, and the presence of C allele at position +2579 (pc<0.0001 for all associations). Similarly, a strong association was observed between the presence of the C, T, A and A alleles at positions +543, +1922, +2043 and +2579 respectively (pc<0.0001 for all associations). However, these allelic associations were not absolute in all individuals. Using our experimental set-up, we were able to determine which allelic variants occurred together on inherited chromosomes, thus defining individual haplotypes. The combination of the four biallelic polymorphisms can potentially give rise to 16 haplotypes. In the present study, we observed eight haplotypes in our UK populations which we designated with the letters A to H (table 3).

The frequency of the detected genotypes and the frequencies with which the individual haplotypes were detected in the population (phenotype frequency) and gene pool (allele frequency) in the UK control and LTX groups are shown in Table 4. Compared to the control population, we observed a significant reduction (M. C. Peitsch (1996) Biochem Soc Trans 24, 274.) in the number of individuals homozygous for the A haplotype (p=0.018, Odds Ratio (OR)=0.378) in the LTX group. The frequency of the A haplotype was also significantly reduced in the LTX population (p=0.007, rr=0.171) and the LTX gene pool (p=0-003, OR=0.389). In contrast, a significant increase was observed in the frequencies of the F and G haplotypes in the LTX population (p=0.023, OR=8.08) and the LTX gene pool (p=0.023, OR=7.8). Analysis of the frequency of the individual alleles in each of the four polymorphic sites revealed significant increases in the frequency of the rarer alleles in three of the four polymorphic sites in the latex group (Table 5). We observed a significant increase in the frequency of the T allele in position +1922 p=0.0012, OR=2.7), the A allele in position +2043 (p=0.044, OR=1.93), and the A allele in positions +2579 (p=0.008, OR=2.31).

The functional significance of the polymorphisms described in the present study is not yet known. However, there are a number of reasons why at least three of the polymorphisms could be of functionally important. McKenzie et al have identified the existence of two forms of IL-13 (McKenzie A N J, Culpepper J A, Malefyt R D, Briere F, et al. (1993) Proc Natl Acad Sci USA 90: 3735-9; McKenzie A N J, et al. (1993) J. Immunol. 150, 5436). The two forms of IL-13 differ at amino acid residue Gln 98, whose incorporation or absence is thought to be the result of alternative splicing of the Gln 98 codon at the 5′ end of exon 4. The biallelic, intronic polymorphism at position +1922, located only 24nt upstream of the Gln 98 codon, may be important in the regulation of the alternative spliced forms of IL-13. Regarding the +2043 polymorphism, we used the Automated Protein Modeling Server SWISS-MODEL (SWISS-MODEL is an Automated Protein Modelling Server running at the GlaxoWellcome Site (URL) http://www.expasy.ch/swissmod/SWISS-MODEL.html; Peitsch (1995) BioTechnology 13, 658; Peitsch (1996) Biochem Soc Trans 24, 274; Guex & Peitsch (1999) Electrophoresis 18, 2714) to predict the effect of the presence of Gln or Arg at position 130 to the three-dimensional structures of the IL-13 protein both in the presence and absence of Gln at position 98. The model predicted an apparent conformational change in the tertiary structure when Arg at amino acid residue 130 was substituted for Gln; this appeared to be even greater in the absence of Gln at amino acid position 98. Finally, the location of polymorphism +2579 in the 3′ untranslated region of exon 4 could theoretically be involved in the regulation of IL-13 mRNA stability, and thus influence the levels of IL-13 production.

In conclusion, this is the first study to describe the existence of IL-13 single nucleotide polymorphisms and to provide hypothesis-generating evidence that these polymorphisms may be important in the development of allergic conditions such as latex allergy.

Antibody Production Methods

Methods for purification of antigens and antibodies are described in Scopes, R. K. (1993) Protein purfication 3rd Edition, Springer Verlag (ISBN 0-387-94072-3 and 3-540-94072-3). The disclosure of this reference, especially chapters 7 and 9, is incorporated herein by reference.

Antibodies may be produced in a number of ways.

1 The protein is purified from the same species as the immunization animal but will usually be human. For monoclonal antibodies, the anion is normally a mouse; for polyclonal, a rabbit or goat.

2. Raise antibodies to the antigen. For polyclonal antibodies, this is simply a matter of injecting suitably prepared sample into the animal at intervals, and testing its serum for the presence of antibodies (for details, see Dunbar, B. S. & Schwoebel, E. D. (1990) Preparation of polyclonal antibodies. Methods Enymol. 182, 663-670). But it is essential that the antigen (ie. the protein of interest) be as pure as possible. For monoclonal antibodies, the purity of the antigen is relatively unimportant if the screening procedure to detect suitable clones uses a bioassay.

Antibodies can also be produced by molecular biology techniques, with expression in bacterial or other heterologous host cells (Chiswell, D. J. & McCafferty, J. (1992) Phage antibodies: will new “coli-clonal” antibodies replace monoclonal antibodies? Trends Biotechnol. 10: 8084). The purification method to be adopted will depend on the source material (serum, cell culture, bacterial expression culture, etc.) and the purpose of the purification (research, diagnostic investigation, commercial production).

The Major Purification Methods are as Follows:

1. Ammonium sulphate precipitation. The γ-globulins precipitate at a lower concentration than most other proteins, and a concentration of 33% saturation is sufficient. Either dissolve in 200 g ammonium sulphate per litre of serum, or add 0.5 volume (vol) of saturated ammonium sulphate. Stir for 30 minutes, then collect the γ-globulin fraction by centrifugation, redissolve in an appropriate buffer, and remove excess ammonium sulphate by dialysis or gel filtration.

2. Polyethylene glycol precipitation. The low solubility of γ-globulins can also be exploited using PEG. Add 0.1 vol of a 50% solution of PEG 6,000 to the serum, stir for 30 minutes and collect the γ-globulins by centrifugation. Redissolve the precipitate in an appropriate buffer, and remove excess PEG by gel filtration on a column that fractionates in a range with a minimum around 6,000 Da.

3. Isoelectric precipitation. This is particularly suited for IgM molecules, and the precise conditions will depend on the exact properties of the antibody being produced.

4. lon-exchange chromatography. Whereas most serum proteins have low isoelectfic points, γ-globulins are isoelectric around neutrality, depending on the exact properties of the antibody being produced. Adsorption to cation exchangers in a buffer of around pH 6 has been used successfully, with elution with a salt gradient, or even standard saline solution to allow immediate therapeutic use.

5. Hydrophobic chromatography. The low solubility of γ-globulins reflects their relatively hydrophobic character. In the presence of sodium or ammnonium sulphate, they bind to many hydrophobic adsorbents, such as “T-gel” which consists of β-mercaptoethanol coupled to divinol sulphone-activated agarose.

6. Affinity adsorbents. The outer coat protein of Staphylococcus aureus, known as Protein A, is isolated from the bacterial cells, and interacts very specifically and strongly with the invariant region (F_{c}) of immunoglobulins (Kessler, S. W. (1975) Rapid isolation of antigens from cells with a staphylococcal protein A-antibody absorbent: Parameters of the interaction of antibody-antigen complexes with protein A. J Immunol. 115, 1617-1624). Protein A has been cloned, and is available in many different forms, but the most useful is as an affinity column, e.g. comprising protein A coupled to agarose. A mixture containing immunoglobulins is passed through the column, and only the immunoglobulins adsorb. Elution is carried out by lowering the pH; different types of IgG elute at different pHs, and so some trials will be needed each time. The differences in the immunoglobulins in this case are not due so much to the antibody specificity, but due to different types of F_{c} region. Each animal species produces several forms of heavy chain varying in the F_{c} region; for instance, mouse immunoglobulins include subclasses IgG₁, IgG₂ₐ, and IgG₃ all of which behave differently on elution from Protein A.

Some γ-globulins do not bind well to Protein A. To isolate such γ-globulins, an alternative affinity adsorbent such as Protein G from a Streptococcus sp. can be used. This is more satisfactory with immunoglobulins from farm animals such as sheep, goats and cattle, as well as with certain subclasses of mouse and rabbit IgGs.

The most specific affinity adsorbent is the antigen itself. The process of purifying an antibody on an antigen adsorbent is essentially the same as purifying the antigen on an antibody adsorbent. The antigen is coupled to the activated matrix, and the antibody-containing sample applied. Elution requires a process for weakening the antibody-antigen complex. This is particularly useful for purifying a specific antibody from a polyclonal mixture.

Monoclonal antibodies (MAbs) can be prepared to most antigens. The antigen-binding portion may be a part of an antibody (for example a Fab fragment) or a synthetic antibody fragment (for example a single chain Fv fragment [ScFv]). Suitable monoclonal antibodies to selected antigens may be prepared by known techniques, for example those disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982).

Chimaeric antibodies are discussed by Neuberger et al (1988, 8th International Biotechnology Symposium Part 2, 792-799).

Suitably prepared non-human antibodies can be “humanized” in known ways, for example by inserting the CDR regions of mouse antibodies into the framework of human antibodies.

The variable heavy (V_{H}) and variable light (V_{L}) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent parental antibody (Morrison et al (1984) Proc. Natl. Acad. Sci. USA 81, 6851-6855).

That antigenic specificity is conferred by variable domains and is independent of the constant domains is known from experiments involving the bacterial expression of antibody fragments, all containing one or more variable domains. These molecules include Fab-like molecules (Better et al (1988) Science 240, 1041); Fv molecules (Skerra et al (1988) Science 240, 1038); single-chain Fv (ScFv) molecules where the V_{H} and V_{L} partner domains are linked via a flexible oligopeptide (Bird et al (1988) Science 242, 423; Huston et al (1988) Proc. Natl. Acad. Sci. USA 85, 5879) and single domain antibodies (dAbs) comprising isolated V domains (Ward et al (1989) Nature 341, 544). A general review of the techniques involved in the synthesis of antibody fragments which retain their specific binding sites is to be found in Winter & Milstein (1991) Nature 349, 293-299.

By “ScFv molecules” we mean molecules wherein the V_{H} and V_{L} partner domains are linked via a flexible oligopeptide.

The advantages of using antibody fragments, rather than whole antibodies, are several-fold. The smaller size of the fragments may lead to improved pharmacological properties, such as better penetration of solid tissue. Effector functions of whole antibodies, such as complement binding, are removed. Fab, Fv, ScFv and dAb antibody fragments can all be expressed in and secreted from E. coli, thus allowing the facile production of large amounts of the said fragments.

Whole antibodies, and F(ab′)₂ fragments are bivalent. By “bivalent” we mean that the said antibodies and F(ab′)₂ fragments have two antigen combining sites. In contrast, Fab, Fv, ScFv and dAb fragments are monovalent, having only one antigen combining sites.

A CDR-grafted antibody may be produced having at least one chain wherein the framework regions are predominantly derived from a first antibody (acceptor) and at least one CDR is derived from a second antibody (donor), the CDR-grafted antibody being capable of binding to the β-form PrP antigen.

The CDR-grafted chain may have two or all three CDRs derived from the donor antibody.

Advantageously, in the CDR-grafted chain, the or each CDR comprises a composite CDR comprising all the residues from the CDR and all the residues in the corresponding hypervariable region of the donor antibody.

Preferably, at least one residue in the framework regions of the CDR-grafted chain has been altered so that it corresponds to the equivalent residue in the antibody, and the framework regions of the CDR-grafted chain are derived from a human antibody.

Advantageously, the framework regions of the CDR-grafted chain are derived from a human Ig heavy chain. For such heavy chains, it is preferred that residue 35 in the heavy chain framework regions be altered so that it corresponds to the equivalent residue in the donor antibody.

Suitably, for such heavy chains, at least one composite CDR comprising residues 26 to 35, 50 to 65 or 95 to 102 respectively is grafted onto the human framework. It will be appreciated in this case that residue 35 will already correspond to the equivalent residue in the donor antibody.

Preferably, residues 23, 24 and 49 in such heavy chains correspond to the equivalent residues in the antibody. It is more preferred that residues 6, 23, 24, 48 and 49 in such heavy chains correspond to the donor antibody in equivalent residue positions. If desired, residues 71, 73 and 79 can also so correspond.

To further optimise affinity, any one or any combination of residues 57, 58, 60, 88 and 91 may correspond to the equivalent residue in the donor antibody.

The heavy chain may be derived from the human KOL heavy chain. However, it may also be derived from the human NEWM or EU heavy chain.

Alternatively, the framework regions of the CDR-grafted chain may be derived from a human kappa or lambda light chain. For such a light chain, advantageously at least one composite CDR comprising residues 24 to 34, 50 to 56 or 89 to 97 respectively is grafted onto the human framework. Preferably, residue 49 also corresponds to the equivalent residue in the donor antibody.

To further optimise affinity, it is preferable to ensure that residues 49 and 89 correspond to the equivalent residues in the donor antibody. It may also be desirable to select equivalent donor residues that form salt bridges.

The light chain is preferably derived from the human REI light chain. However, it may also be derived from the human EU light chain.

Preferably, the CDR-grafted antibody comprises a light chain and a heavy chain, one or, preferably, both of which have been CDR-grafted in accordance with the principles set out above for the individual light and heavy chains.

It is advantageous that all three CDRs on the heavy chain are altered and that minimal alteration is made to the light chain. It may be possible to alter none, one or two of the light chain CDRs and still retain binding affinity at a reasonable level.

It will be appreciated that in some cases, for both heavy and light chains, the donor and acceptor residues may be identical at a particular position and thus no change of acceptor framework residue will be required.

It will also be appreciated that in order to retain as far as possible the human nature of the CDR-grafted antibody, as few residue changes as possible should be made. It is envisaged that in many cases, it will not be necessary to change more than the CDRs and a small number of framework residues. Only in exceptional cases will it be necessary to change a larger number of framework residues.

Preferably, the CDR-grafted antibody is a complete Ig, for example of isotype IgG₁, or IgG₂, IgG₃ or IgM.

If desired, one or more residues in the constant domains of the Ig may be altered in order to alter the effector functions of the constant domains.

Preferably, the CDR-grafted antibody has an affinity for the protein of the second aspect of the invention antigen of between about 10⁵.M⁻¹ to about 10¹².M⁻¹, more preferably at least 10⁸.M-⁻¹.

Advantageously, the one or more CDR is derived from a mammalian antibody and preferably is derived from a murine MAb.

Suitably, the CDR-grafted antibody is produced by use of recombinant DNA technology.

A further method for producing a CDR-grafted antibody comprises providing a first DNA sequence, encoding a first antibody chain in which the framework regions are predominantly derived from a first antibody (acceptor) and at least one CDR is derived from a second antibody (acceptor), under the control of suitable upstream and downstream elements; transforming a host cell with the first DNA sequence; and culturing the transformed host cell so that a CDR-grafted antibody is produced.

Preferably, the method further comprises: providing a second DNA sequence, encoding a second antibody chain complementary to the first chain, under the control of suitable upstream and downstream elements; and transforming the host cell with both the first and second DNA sequences.

Advantageously, the second DNA sequence encodes a second antibody chain in which the framework regions are predominantly derived from a first antibody (acceptor) and at least one CDR is derived from the second antibody (donor).

The first and second DNA sequences may be present on the same vector. In this case, the sequences may be under the control of the same or different upstream and/or downstream elements.

Alternatively, the first and second DNA sequences may be present on different vectors.

A nucleotide sequence may be formed which encodes an antibody chain in which the framework regions are predominantly derived from a first antibody (acceptor) and at least one CDR is derived from a second antibody (donor), the antibody chain being capable of forming a CDR-grafted antibody.

The CDR-grafted antibodies may be produced by a variety of techniques, with expression in transfected cells, such as yeast, insect, CHO or myeloma cells, being preferred. Most preferably, the host cell is a CHO host cell.

To design a CDR-grafted antibody, it is first necessary to ascertain the variable domain sequence of an antibody having the desired binding properties. Suitable source cells for such DNA sequences include avian, mammalian or other vertebrate sources such as chickens, mice, rats and rabbits, and preferably mice. The variable domain sequences (V_{H} and V_{L}) may be determined from heavy and light chain cDNA, synthesized from the respective mRNA by techniques generally known to the art. The hypervariable regions may then be determined using the Kabat method (Wu and Kabat, J. (1970) J. Exp. Med. 132, 211). The CDRs may be determined by structural analysis using X-ray crystallography or molecular is modelling techniques. A composite CDR may then be defined as containing all the residues in one CDR and all the residues in the corresponding hypervariable region. These composite CDRs along with certain select residues from the framework region are preferably transferred as the “antigen binding sites”, while the remainder of the antibody, such as the heavy and light chain constant domains and remaining framework regions, may be based on human antibodies of different classes. Constant domains may be selected to have desired effector functions appropriate to the intended use of the antibody so constructed. For example, human IgG isotypes, IgG₁ and IgG₃ are effective for complement fixation and cell mediated lysis. For other purposes other isotypes, such as IgG₂ and IgG₄, or other classes, such as IgM and IgE, may be more suitable.

For human therapy, it is particularly desirable to use human isotypes, to minimise antiglobulin responses during therapy. Human constant domain DNA sequences, preferably in conjunction with their variable domain framework bases can be prepared in accordance with well-known procedures. An example of this is CAMPATH 1H available from Glaxo Wellcome.

Certain CDR-grafted antibodies are provided which contain select alterations to the human-like framework region (in other words, outside of the CDRs of the variable domains), resulting in a CDR-grafted antibody with satisfactory binding affinity. Such binding affinity is preferably from about 10⁵.M⁻¹ to about 10¹².M⁻¹ and is more preferably at least about 10⁸.M⁻¹.

In constructing the CDR-grafted antibodies, the V_{H} and/or V_{L} gene segments may be altered by mutagenesis. One skilled in the art will also understand that various other nucleotides coding for amino acid residues or sequences contained in the Fc portion or other areas of the antibody may be altered in like manner (see, for example, PCT/US89/00297).

Exemplary techniques include the addition, deletion or nonconservative substitution of a limited number of various nucleotides or the conservative substitution of many nucleotides, provided that the proper reading frame is maintained.

Substitutions, deletions, insertions or any subcombination may be used to arrive at a final construct. Since there are 64 possible codon sequences but only twenty known amino acids, the genetic code is degenerate in the sense that different codons may yield the same amino acid. Thus there is at least one codon for each amino acid, i.e. each codon yields a single amino acid and no other. It will be apparent that during translation, the proper reading frame must be maintained in order to obtain the proper amino acid sequence in the polypeptide ultimately produced.

Techniques for additions, deletions or substitutions at predetermined amino acid sites having a known sequence are well known. Exemplary techniques include oligonucleotide-mediated site-directed mutagenesis and the polymerase chain reaction.

Oligonucleotide site-directed mutagenesis in essence involves hybridizing an oligonucleotide coding for a desired mutation with a single strand of DNA containing the region to be mutated and using the single strand as a template for extension of the oligonucleotide to produce a strand containing the mutation. This technique, in various forms, is described in Zoller and Smith (1982) Nucl. Acids Res. 10, 6487.

Polymerase chain reaction (PCR) in essence involves exponentially amplifying DNA in vitro using sequence specific oligonucleotides. The oligonucleotides can incorporate sequence alterations if desired. The polymerase chain reaction technique is described in Mullis and Fuloona (1987) Meth. Enz. 155, 335. Examples of mutagenesis using PCR are described in Ho et al (1989) Gene 77, 51.

The nucleotide sequences, capable of ultimately expressing the desired CDR-grafted antibodies, can be formed from a variety of different polynucleotides (genomic DNA, cDNA, RNA or synthetic oligonucleotides). At present, it is preferred that the polynucleotide sequence comprises a fusion of cDNA and genomic DNA. The polynucleotide sequence may encode various Ig components (eg V, J, D, and C domains). They may be constructed by a variety of different techniques. Joining appropriate genomic and cDNA sequences is presently the most common method of production, but cDNA sequences may also be utilized (see EP-A-0 239 400).

Raising an Antibody Response in a Patient

Active immunisation of the patient is preferred. In this approach, the protein is prepared in an immunogenic formulation containing suitable adjuvants and carriers and administered to the patient. Suitable adjuvants include Freund's complete or incomplete adjuvant, muramyl dipeptide, the “Iscoms” of EP 109 942, EP 180 564 and EP 231 039, aluminium hydroxide, saponin, DEAE-dextaan, neutral oils (such as miglyol), vegetable oils (such as arachis oil), liposomes, Pluronic polyols or the Ribi adjuvant system (see, for example GB-A-2 189 141). “Pluronic” is a Registered Trade Mark.

It may be advantageous to use a protein from a species other than the one being treated, in order to provide for a greater immunogenic effect.

Punfication of Antigens and Antibodies by Affinity Chromatography

Antigen or antibody is bound through its free amino groups to cyanogen-bromide-activated Sepharose particles. Insolubilized antibody, for example, can be used to pull the corresponding antigen out of solution in which it is present as one component of a complex mixture, by absorption to its surface. The unwanted material is washed away and the required ligand released from the affinity absorbent by disruption of the antigen-antibody bonds by changing the pH or adding chaotropic ions such as thiocyanate. Likewise, an antigen inununosorbent can be used to absorb out an antibody from a mixture whence it can be purified by elution. The potentially damaging effect of the eluting agent can be avoided by running the anti-serum down an affinity column so prepared as to have relatively weak binding for the antibody being purified; under these circumstances, the antibody is retarded in flow rate rather than being firmly bound. If a protein mixture is separated by iso-electric focusing into discrete bands, an individual band can be used to affinity purify specific antibodies from a polyclonal antiserum.

[image]

Immunoassay of Antigen and Antibody with Labelled Reagents

Antigen and antibody can be used for the detection of each other and a variety of immunoassay techniques have been developed in which the final read-out of the reaction involves a reagent conjugated with an appropriate label. Radiolabelling with ¹³¹I, ¹²⁵I, is an established technique.

Soluble Phase Immunoassays

Radioimmunoassay (RIA) for Antigen

The binding of radioactively labelled antigen to a limited fixed amount of antibody can be partially inhibited by addition of unlabelled antigen and the extent of this inhibition can be used as a measure of the unlabelled material added.

For Antibody

The antibody content of a serum can be assessed by the ability to bind to antigen which has been in and immobilised by physical absorption to a plastic tube or micro-agglutination tray with multiple wells; the bound immunoglobin may then be estimated by addition of a labelled anti-Ig raised for anther species. For example, a patient's serum is added to a microwell coated with antigen, the antibodies will bind to the plastic and remaining serum proteins can be readily washed away. Bound antibody can be estimated by addition of ¹²⁵I-labelled purified rabbit anti IgG; after rinsing out excess unbound reagent, the radioactivity of the rube will be a measure of the antibody content of the patient's serum. The distribution of antibody in different classes can obviously be determined by using specific antisera.

[image]

Immunoradiometric Assay for Antigen

This differs from radioimmunoassay in the sense that the labelled reagent is used in excess. For the estimation of antigen, antibodies are coated on to a solid surface such as plastic and the test antigen solution added; after washing, the amount of antigen bound to the plastic can be estimated by adding an excess of radio-labelled antibody. The specificity of the method can be improved by the sandwich assay, which uses solid phase and labelled antibodies with specificities for different parts of the antigen:

[image]

Because of health hazards and the deterioration of reagents through radiation damage, types of label other than radioisotopes have been sought.

ELISA (Enzyme-Linked Immunosorbent Assay)

Perhaps the most widespread alternative has been the use of enzymes which give a coloured reaction product, usually in solid phase assays. Enzymes such as horse radish peroxidase and phosphatase have been widely employed. A way of amplifying the phosphatase reaction is to use NADP as a substrate to generate NAD which now acts as a coenzyze for a second enzyme system. Pyrophosphatase from E. coli provides a good conjugate because the enzyme is not present in tissues, is stable and gives a good reaction colour. Chemi-luminescent systems based on enzymes such as luciferase can also be used.

Conjugation with the vitamin biotin is frequently used since this can readily be detected by its reaction with enzyme-linked avidin or streptavidin to which it binds with great specificity and affinity.

Identification of Ligands by Phage Display

The display of proteins and polypeptides on the surface of bacteriophage (phage), fused to one of the phage coat proteins, provides a powerful tool for the selection of specific ligands. This ‘phage display’ technique was originally used by Smith (1985) Science 228, 1315-7 to create large libraries of antibodies for the purpose of selecting those with high affinity for a particular antigen. More recently, the method has been employed to present peptides, domains of proteins and intact proteins at the surface of phages in order to identify ligands having desired properties.

The principles behind phage display technology are as follows:

(i) Nucleic acid encoding the protein or polypeptide for display is cloned into a phage;

(ii) The cloned nucleic acid is expressed fused to the coat-anchoring part of one of the phage coat proteins (typically the p3 or p8 coat proteins in the case of filamentous phage), such that the foreign protein or polypeptide is displayed on the surface of the phage;

(iii) The phage displaying the protein or polypeptide with the desired properties is then selected (e.g. by affinity chromatography) thereby providing a genotype (linked to a phenotype) that can be sequenced, multiplied and transferred to other expression systems.

Alternatively, the foreign protein or polypeptide may be expressed using a phagemid vector (i.e. a vector comprising origins of replication derived from a phage and a plasmid) that can be packaged as a single stranded nucleic acid in a bacteriophage coat. When phagemid vectors are employed, a “helper phage” is used to supply the functions of replication and packaging of the phagemid nucleic acid. The resulting phage will express both the wild type coat protein (encoded by the helper phage) and the modified coat protein (encoded by the phagemid), whereas only the modified coat protein is expressed when a phage vector is used.

Methods of selecting phage expressing a protein or peptide with a desired specificity are known in the art. For example, a widely used method is “panning”, in which phage stocks displaying ligands are exposed to solid phase coupled target molecules, e.g. using affinity chromatography. Alternative methods of selecting phage of interest include SAP (Selection and Amplification of Phages; as described in WO 95/16027) and SIP (Selectively-lnfective Phage; EP 614989A, WO 99/07842), which employ selection based on the amplification of phages in which the displayed ligand specifically binds to a ligand binder. In one embodiment of the SAP method, this is achieved by using non-infectious phage and connecting the ligand binder of interest to the N-terminal part of p3. Thus, if the ligand binder specifically binds to the displayed ligand, the otherwise non-infective ligand-expressing phage is provided with the parts of p3 needed for infection. Since this interaction is reversible, selection can then be based on kinetic parameters (see Duenas et al., 1996, Mol. Immunol. 33, 279-285).

The use of phage display to isolate ligands that bind biologically relevant molecules has been reviewed in Felici et al. (1995) Biotechnol. Annual Rev. 1, 149-183, Katz (1997) Annual Rev. Biophys. Biomol. Struct. 26, 27-45 and Hoogenboom et al. (1998) Immunotechnology 4(1), 1-20. Several randomised combinatorial peptide libraries have been constructed to select for polypeptides that bind different targets, e.g. cell surface receptors or DNA (reviewed by Kay, 1995, Perspect. Drug Discovery Des. 2, 251-268; Kay and Paul, 1996, Mol. Divers. 1, 139-140). Proteins and multimeric proteins have been successfully phage-displayed as functional molecules (see EP 0 349 578 A, EP 0 527 839 A, EP 0 589 877 A; Chiswell and McCafferty, 1992, Trends Biotechnol. 10, 80-84). In addition, functional antibody fragments (e.g. Fab, single chain Fv [scFv]) have been expressed (McCafferty et al., 1990, Nature 348, 552-554; Barbas et al., 1991, Proc. Natl. Acad. Sci. USA 88, 7978-7982; Clackson et al., 1991, Nature 352, 624-628), and some of the shortcomings of human monoclonal antibody technology have been superseded since human high affinity antibody fragments have been isolated (Marks et al., 1991, J. Mol. Biol. 222, 581-597; Hoogenboom and Winter, 1992, J. Mol. Biol. 227, 381-388). Further information on the principles and practice of phage display is provided in Phage display of peptides and proteins: a laboratory manual Ed Kay, Winter and McCafferty (1996) Academic Press, Inc ISBN 0-12-402380-0, the disclosure of which is incorporated herein by reference.

1. An isolated nucleic acid molecule having a variation of the IL-13 encoding sequence shown in FIG. 1 [SEQ ID No 1]; wherein the variation is at least one of G to C at position +543nt and/or C to T at position +1922nt and/or G to A at position +2043nt and/or C to A at position +2579nt upstream of the initiation codon.

2. A molecule as claimed in claim 1 wherein the variation is G to A at position +2043nt.

3. A molecule as claimed in claim 1, wherein the variation is C to T at position +1922; G to A at position +2043 and C to A at position +2579.

4. An isolated amino acid sequence encoded by a nucleic acid molecule according to any one of claims 1 to 3 and comprising glutamine at an amino acid position corresponding to position 130 of the unprocessed precursor.

5. An isolated amino acid sequence according to claim 4 having IL-13 activity.

6. Use of an amino acid sequence as claimed in claim 4 or 5 in a method of producing an antibody.

7. Use as claimed in claim 6 wherein the antibody is a polyclonal antibody.

8. Use as claimed in claim 6, wherein the antibody is a monoclonal antibody.

9. An antibody obtainable by a use as claimed in any one of claims 6 to 8 wherein the antibody specifically binds the IL-13 amino acid sequence of claim 5 and does not exhibit significant cross-reactivity with a different IL-13 encoding amino acid sequence.

10. A nucleic acid molecule as claimed in any one of claims 1 to 3 for use in medicine.

11. An amino acid sequence as claimed in claim 4 or 5 for use in medicine.

12. A transgenic, non-human mammalian animal whose germ cells and somatic cells contain a nucleic acid molecule according to any one of claims 1 to 3.

13. A transgenic animal according to claim 12 capable of expressing an amino acid sequence having IL-13 activity and containing glutamine at amino acid position 130.

14. A method of producing a transgenic, non-human mammalian animal according to claim 12 or 13, said method comprising introducing a nucleic acid molecule according to any one of claims 1 to 3 into the genome of a non-human mammalian animal, preferably at a stage no later than the 8-cell stage.

15. A method of detecting susceptibility or resistance to a disorder associated with expression of IL-13 comprising testing nucleic acid from an individual for the presence or absence of a variation in the nucleotide sequence encoding IL-13 as defined in any one of claims 1 to 3.

16. A method of detecting susceptibility or resistance to a disorder associated with expression of IL-13 comprising testing a biological sample from an individual for the presence or absence of an amino acid sequence as defined in claim 4 or 5.

17. A method of detecting susceptibility or resistance to a disorder associated with an immune response comprising testing nucleic acid from an individual for the presence of a variation in the nucleotide sequence encoding IL-13 as defined in any one of claims 1 to 3.

18. A method of detecting susceptibility or resistance to latex sensitisation of an individual comprising testing nucleic acid from the individual for the presence or absence of a variation in the nucleotide sequence encoding IL-13 as defined in any one of claims 1 to 3, the presence of such a variation being indicative of latex sensitivity.

19. A method as claimed in any one of claims wherein the amino acid sequence is detected using an antibody.

20. A method as claimed in any one of claims 15 to 17 wherein the disorder is associated with an immune response and is preferably asthma and/or latex sensitisation.

21. An antibody obtainable by use or method as claimed in any one of claims 6 to 8 for use in medicine.

22. A method of treatment of a patient with an immune response disorder comprising administering to said patient a blocking agent which binds to a nucleic acid molecule according to any one of claims 1 to 3 and/or an amino acid sequence according to claim 4 or 5, thereby preventing or reducing expression of said nucleic acid molecule and/or preventing or reducing a function of said amino acid sequence.

23. A method according to claim 22 wherein the blocking agent is an antisense oligonucleotide.

24. A method according to claim 23 wherein the blocking agent is an antibody according to claim 9.

25. A method according to any one of claims 22 to 24 wherein the patient suffers from asthma, atopic allergy and/or latex sensitisation.


SEQUENCE LISTING (17 sequences)

SEQ ID NO: 1 - DNA, 4600 bp, Homo sapiens
cctaggcaggcaacatagtgagaccccatctccaaaaaaacaaaacaaaacaaaacaaaaaaacaccaaaaaagctcccagaaagacctctgaatctttctggatctctcagtggagaccttggaaatctgaactttgacaatccctctcacagtggggccaaggaggaattaggcaagccaaaagaagtgaactttactcttctattgcctgtttgaattttgtatccaagcaagtgttacttaagtaatttaagagactggttcatcgaaaaaataaaactccccaaattcccatagctggtagactgtggtcacagccacagtgcactaagactatctgctcagcacttctggtgacccaaaagggtctgaggacaggagctcagagttgggtcagctgtccaggtactcagggttgtcacaggcaaaactgctggaactcagggcagcattgcaaatgcctcgccgctctcgaggccccttgcctgccgctggaattaaacccacccagatcttggaaactctgccctggacccttctcaataagtccatgagaaatcaaactctttcctttatgcgacactggattttccacaaagtaaaatcaagatgagtaaagatgtggtttctagatagtgcctgaaaaagcagagaccatggtgtcaggcgtcaccacttgggcctataaaagctgccacaagagcccaagccacaagccacccagcctatgcatccgctcctcaatcctctcctgttggcactgggcctcatggcgcttttgttgaccacggtcattgctctcacttgccttggcggctttgcctccccaggccctgtgcctccctctacagccctcagggagctcattgaggagctggtcaacatcacccagaaccagaaggtgagtgtcggctagccagggtcctagctatgagggctccagggtgggtgattcccaagatgaggtcatgagcaggctgggcctggtcctaagatgcctgtaggtcaggaaaaatctccatggaccaaggcccggcccagccatgagggagagaggagctgggctggggggctcagcactgtggatggacctatggaggtgtctggcagactccccagggactacctgctctcctggcctggccttgtctgccactgccagctcctactcagccattcctgaacagaggacagcagagaagggtccagcaccctcccagaaccatgtggcatttgccaactggattttgaccataacaatgcagccattctccccagcaccatcataggcccgcccttacaggaggattcgttagtagagtccgctccttgccccactagtaacagctcacatgtctgagcactgcttacaccaggcctggtgcacgtgctttatgtgtcatttcatcactgccagccacctcaagaggcaggtacgatgaacccattctgctaaggttcagtgaggttaagtgacagaggctggattcaagccaggcctggccaacaccagagtgtccatgctcctaactgcagtgttccctcaccatcagaaggcagggcatttaatacaccagatccccaccgcctcccatctgatttgtcttggtcaacagtggcccaggccatcctacttcactcgtccccaccctggcccttcccgcagcccctgtcctcctgccctgactatggcaagccttgcatgcagcttgtcccttactagtggtgtcaatttttttctctcagctccaagaccctaaacagtgggacctcacccctatgcctgctgttcaaagcagaaaacgaagctcaggaatgctgaggggctgccaggcctgcctctgtgccacaccagggatgcttgtggggcctgtgctggggcagacctggcctgggctgccagggcaggcccacaacccctgccagcactctgctcactgtcactttgctcccacaggctccgctctgcaatggcagcatggtatggagcatcaacctgacagctggcatggtaaggacctttgggtgcagggaggatggggcagaggctccaggccttgggcttatcttctctgagcctcccttccatggctggggttccaagcaagcttcaagtgctctcctccctcccgccataatctggccccttcccgcccaccacccagactcacctgcgccaggcatctcagccccatcttcctgcagactcacaaaaggcagctgcccaagcagggcctgacccctcggtgtcccctccccacagtactgtgcagccctggaatccctgatcaacgtgtcaggctgcagtgccatcgagaagacccagaggatgctgagcggattctgcccgcacaaggtctcagctggggtaaggcatcccccaccctctcacacccaccctgcaccccctcctgccaaccctgggctcgctgaagggaagctggctgaatatccatggtgtgtgtccacccaggggtggggccattgtggcagcagggacgtggccttcgggatttacaggatctgggctcaagggctcctaactcctacctgggcctcaatttccacatctgtacagtagaggtactaacagtacccacctcatggggacttccgtgaggactgaatgagacagtccctggaaagcccctggtttgtgcgagtcgtcccggcctctggcgttctactcacgtgctgacctctttgtcctgcagcagttttccagcttgcatgtccgagacaccaaaatcgaggtggcccagtttgtaaaggacctgctcttacatttaaagaaactttttcgcgagggacggttcaactgaaacttcgaaagcatcattatttgcagagacaggacctgactattgaagttgcagattcatttttctttctgatgtcaaaaatgtcttgggtaggcgggaaggagggttagggaggggtaaaattccttagcttagacctcagcctgtgctgcccgtcttcagcctagccgacctcagccttccccttgcccagggctcagcctggtgggcctcctctgtccagggccctgagctcggtggacccagggatgacatgtccctacacccctcccctgccctagagcacactgtagcattacagtgggtgccccccttgccagacatgtggtgggacagggacccacttcacacacaggcaactgaggcagacagcagctcaggcacacttcttcttggtcttatttattattgtgtgttatttaaatgagtgtgtttgtcaccgttggggattggggaagactgtggctgctggcacttggagccaagggttcagagactcagggccccagcactaaagcagtggaccccaggagtccctggtaataagtactgtgtacagaattctgctacctcactggggtcctggggcctcggagcctcatccgaggcagggtcaggagaggggcagaacagccgctcctgtctgccagccagcagccagctctcagccaacgagtaatttattgtttttcctcgtatttaaatattaaatatgttagcaaagagttaatatatagaagggtaccttgaacactgggggaggggacattgaacaagttgtttcattgactatcaaactgaagccagaaataaagttggtgacagataggcctgattgtatttgtctttcattttggcctttggggacactggtctgtggtctgaagactctgaggagctcttcgggaggctggtgggttggaggaggggactgggatggattacagcgagggtagggtgcagtgacctgggctgaatgcaagctagctcccgagggtggggacatggcctgaaggaagccccaccttctgtctgctgcaccagcaaggacggagaggcttgggcagactgtcagggttcaaggagggcatcaggagcagacggagacccaggaagtctcacaatcacatctcctgaggactggccagctgtgtctggcaccacccacacatccatgtctccctcacaacccaggaggccgatgagaactgtgaggctcagaaagcgtgggcggtttgcctaaggtcacgtagctacttcctcactggggtcctggggcctcagagcctcatctgaggtaaaggagcaaagttgggattggggtccaaaattcactttaactccaaagcccacacacttaaccaccctgcctatttctgtccaaatgtcacctgtcctgaatggagtttttccccctgtacaactgtcatcaacctgttcgggccctctcactgacaggcaggtccctacctatatttgaggggcagcccattgcatttctggacagctctcgccattagggtgcacacacgcaccacctctgtgaacagggctctggctaggccactcctcagcagctcttgttgcttccccatggccctggtcagcagctggagtgcagagaccagcgggccttaccaagccacagctccaggccatgccgtcagcaacacttttcactgtgactctctgggaggtgcccagggcagagggtgactccaggatgggatgcctttgcagtgggtgatggtctttcaagttccagtctcaaacttg

SEQ ID NO: 2 - protein, 132 aa, Homo sapiens
MALLLTTVIALTCLGGFASPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGQFN

SEQ ID NO: 3 - DNA, 19 bp, Artificial Sequence
  PCR Primer
gcccttacaggaggattcg

SEQ ID NO: 4 - DNA, 19 bp, Artificial Sequence
  PCR Primer
gcccttacaggaggattcc

SEQ ID NO: 5 - DNA, 18 bp, Artificial Sequence
  PCR Primer
gccattgcagagcggagc

SEQ ID NO: 6 - DNA, 20 bp, Artificial Sequence
  PCR Primer
gcctctggcgttctactcat

SEQ ID NO: 7 - DNA, 19 bp, Artificial Sequence
  PCR Primer
cctctggcgttctactcac

SEQ ID NO: 8 - DNA, 23 bp, Artificial Sequence
  PCR Primer
gctttcgaagtttcagttgaact

SEQ ID NO: 9 - DNA, 23 bp, Artificial Sequence
  PCR Primer
gctttcgaagtttcagttgaacc

SEQ ID NO: 10 - DNA, 21 bp, Artificial Sequence
  PCR Primer
ttattaccagggactcctggt

SEQ ID NO: 11 - DNA, 19 bp, Artificial Sequence
  PCR Primer
attaccagggactcctggg

SEQ ID NO: 12 - DNA, 20 bp, Artificial Sequence
  PCR Primer
aggacaaagaggtcagcacg

SEQ ID NO: 13 - DNA, 20 bp, Artificial Sequence
  PCR Primer
aggacaaagaggtcagcaca

SEQ ID NO: 14 - DNA, 19 bp, Artificial Sequence
  PCR Primer
tgccaagtggagcacccaa

SEQ ID NO: 15 - DNA, 20 bp, Artificial Sequence
  PCR Primer
gcatcttgctctgtgcagat

SEQ ID NO: 16 - DNA, 21 bp, Artificial Sequence
  PCR Primer
atgatgttgacctttccaggg

SEQ ID NO: 17 - DNA, 23 bp, Artificial Sequence
  PCR Primer
ttctgtaacttttcatcagttgc